(19) United States
(12) Patent Application Publication
(10) Pub. No.: US 2004/0039023 A1
(43) Pub. Date: Feb. 26, 2004

(54) Therapeutic agents

(76) Inventors: Alan Martin Birch, Nottingham (GB); Patricia Lesley Needham, Nottingham (GB)

Correspondence Address: BROMBERG & SUNSTEIN LLP, 125 SUMMER STREET, BOSTON, MA, 02110-1618, US

(21) Appl. No.: 10/240,180

(22) PCT Filed: Mar. 27, 2001
(86) PCT No.: PCT/EP01/03463

(30) Foreign Application Priority Data
Mar. 28, 2000 (GB) 0007376.7

(51) Int. Cl. A61K 31/452; C 07D 4 7/02

(52) U.S. Cl. 514/321; 546/197

(57) ABSTRACT

Compound of formula (I) including pharmaceutically acceptable salts thereof in which R₁ represents halo or pseudohalo and R₂ represents H or an acyl group derived from a C₇-C₁₈ saturated aliphatic carboxylic acid; with the proviso that when R₁ is Cl or CF₃ and R₂ is H then these compounds are in isolated form, their preparation and their use in the treatment of depression, anxiety, psychoses, Parkison's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders, anorexia, cardiovascular and cerebrovascular disorders, non-insulin dependent diabetes mellitus, hyperglycaemia, constipation, arrhythmia, disorders of the neuroendocrine system, stress, prostatic hypertrophy, drug-induced extrapyramidal symptoms or spasticity; are described.

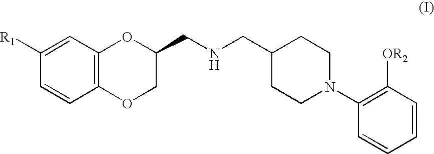

The present invention relates to novel therapeutic agents which have affinity for 5-HT_{1A} and/or α₁ and/or D₂ receptors, to processes for their preparation, to pharmaceutical compositions containing them and to their use in the treatment of central nervous system disorders, for example depression, anxiety, psychoses (for example schizophrenia), tardive dyskinesia, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders and anorexia, cardiovascular and cerebrovascular disorders, migraine, non-insulin dependent diabetes mellitus, hyperglycaemia, constipation, arrhythmia, disorders of the neuroendocrine system, stress, prostatic hypertrophy, drug-induced extrapyramidal symptoms and spasticity.

In WO93/17017 there are described [(benzodioxanyl, benzofuranyl and benzopyranyl)alkylamino]alkyl substituted 2-pyrimidinyl compounds which have vasoconstrictor activity. These compounds are claimed to be useful in treating conditions related to vasodilation.

In WO95/07274 compounds of formula I

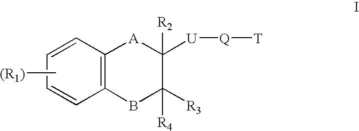

and pharmaceutically acceptable salts thereof in which A is methylene or —O—; B is methylene or —O—; and g is 0, 1, 2, 3 or 4; R₁ represents, halo, optionally substituted alkyl, optionally substituted alkoxy, optionally substituted alkylthio, hydroxy, acyloxy, hydroxymethyl, cyano, alkanoyl, alkoxycarbonyl, optionally N-substituted carbamoyl, carbamoylmethyl, sulphamoyl or sulphamoylmethyl, an amino group optionally substituted by one or two alkyl groups, or two adjacent R₁ groups together with the carbon atoms to which they are attached form a fused benz ring; R₂ is H, alkyl or alkoxy; R₃ and R₄, which are the same or different, are H, or alkyl; U is analkylene chain optionally substituted by one or more alkyl; Q represents a divalent group of formula IIa, IIb or IIc

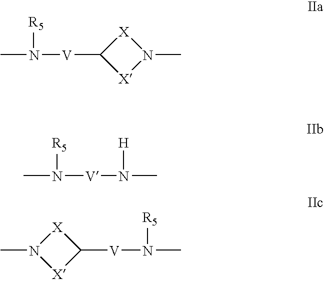

in which V is a bond or an alkylene chain optionally substituted by one or more alkyl; VN is an alkylene chain optionally substituted by one or more alkyl; X is a bond or an alkylene chain and X′ is an alkylene chain, provided that the total number and carbon atoms in X and X′ amounts to 3 or 4; R₅ is H, or alkyl; and T represents an optionally substituted aromatic group which optionally contains one or more N atoms, provided that T is not 2-pyrimidinyl when A is —O—; are disclosed as having utility in the treatment of central nervous system disorders, for example depression, anxiety, psychoses (for example schizophrenia), tardive dyskinesia, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders and anorexia, cardiovascular and cerebrovascular disorders, non-insulin dependent diabetes mellitus, hyperglycaemia, constipation, arrhythmia, disorders of the neuroendocrine system, stress, prostatic hypertrophy, and spasticity.

WO99/62902 discloses compounds of formula I

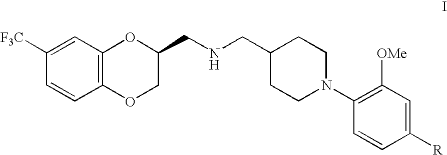

including pharmaceutically acceptable salts thereof in which R represents H or F as a selection invention over WO95/07274.

WO99/62902 disclos s that (S)-(−)-1-[1-(4-fluoro-2-methoxyphenyl)piperid-4-yl]-N-(7-trifluoromethyl-2,3-dihydro-1,4-benzodioxin-2-ylmethyl)methylamine and (S)-(−)-1-[1-(2-methoxyphenyl)piperid-4-yl]-N-(7-trifluoromethyl-2,3-dihydro-1,4-benzodioxin-2-ylmethyl)methylamine have unexpectedly superior activity to the compounds disclosed in WO95/07274.

Surprisingly, it has been found that compounds related to those in WO95/07274, where T is a hydroxyphenyl group, are potently orally active in animal models of psychosis, despite the potential for the hydroxyphenyl group to undergo first pass conjugation.

The present invention provides compounds of formula I

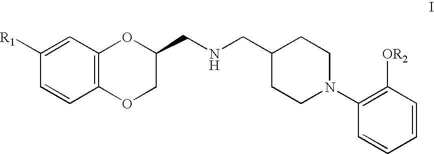

including pharmaceutically acceptable salts thereof in which R₁ represents halo or pseudohalo and R₂ represents H or an acyl group derived from a C₇-C₁₈ saturated aliphatic carboxylic acid; with the proviso that when R₁ is Cl or CF₃ and R₂ is H then these compounds are in isolated form.

The term pseudohalo includes trifluoromethyl, trifluoromethoxy and trifluoromethylthio and other pharmaceutically acceptable pseudohalo groups known to those skilled in the art. Preferably the pseudohalo group is trifluoromethyl, trifluoromethoxy and trifluoromethylthio. The term halo includes chloro, bromo and fluoro.

Preferably R₂ represents H, heptanoyl, decanoyl, dodecanoyl, hexadecanoyl or octadecanoyl. More preferably R₂ is H.

Preferred compounds of formula I are represented by formula II

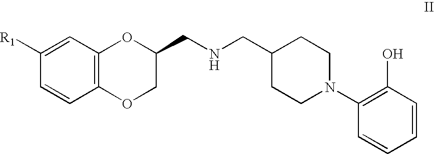

in which R₁ represents Cl or CF₃, including pharmaceutically acceptable salts thereof, in isolated form.

The term isolated is used to indicate that the compounds of the invention are in pure form and are not present in a human or an animal either in a free form or in an associated form.

Specific compounds of the present invention are:

(S)-(−)-2-{4-[N-(7-chloro-2,3-dihydro-1,4-benzodioxin-2ylmethyl)aminomethyl]-piperidino}phenol and

(S)-(−)-2-{4-[N-(7-trifluoromethyl-2,3-dihydro-1,4-benzodioxin-2-ylmethyl)-aminomethyl]piperidino}phenol

and pharmaceutically acceptable salts thereof.

The compounds of the present invention are advantageous over compounds known in the prior art because of their selectivity in receptor binding assays and their superior oral activity.

Compounds of formula I may exist as salts with pharmaceutically acceptable acids. Examples of such salts include hydrochlorides, hydrobromides, sulphates, methanesulphonates, nitrates, maleates, acetates, citrates, fumarates, tartrates [eg (+)-tartrates, (−)-tartrates or mixtures thereof including racemic mixtures], succinates, benzoates and salts with amino acids such as glutamic acid. Compounds of formula I and their salts may exist in the form of solvates (for example hydrates).

Certain compounds of formula I and their salts may exist in more than one crystal form and the present invention includes each crystal form and mixtures thereof. Certain compounds of formula I and their salts may also exist in the form of solvates, for example hydrates, and the present invention includes each solvate and mixtures thereof.

The present invention also includes pharmaceutical compositions containing a therapeutically effective amount of a compound of formula I or a salt thereof together with a pharmaceutically acceptable diluent or carrier.

As used hereinafter, the term active compound denotes a compound of formula I or a salt thereof. In therapeutic use, the active compound may be administered orally, rectally, parenterally or topically, preferably orally. Thus the therapeutic compositions of the present invention may take the form of any of the known pharmaceutical compositions for oral, rectal, parenteral or topical administration. Pharmaceutically acceptable carriers suitable for use in such compositions are well known in the art of pharmacy. The compositions of the invention may contain 0.1-99% by weight of active compound. The compositions of the invention are generally prepared in unit dosage form. Preferably the unit dosage of active ingredient is 1-500 mg. The excipients used in the preparation of these compositions are the excipients known in the pharmacist's art.

Compositions for oral administration are the preferred compositions of the invention and these are the known pharmaceutical forms for such administration, for example tablets, capsules, syrups and aqueous or oil suspensions. The excipients used in the preparation of these compositions are the excipients known in the pharmacist's art. Tablets may be prepared by mixing the active compound with an inert diluent such as calcium phosphate in the presence of disintegrating agents, for example maize starch, and lubricating agents, for example magnesium stearate, and tableting the mixture by known methods. The tablets may be formulated in a manner known to those skilled in the art so as to give a sustained release of the compounds of the present invention. Such tablets may, if desired, be provided with enteric coatings by known methods, for example by the use of cellulose acetate phthalate. Similarly, capsules, for example hard or soft gelatin capsules, containing the active compound with or without added excipients, may be prepared by conventional means and, if desired, provided with enteric coatings in a known manner. The tablets and capsules may conveniently each contain 1 to 500 mg of the active compound. Other compositions for oral administration include, for example, aqueous suspensions containing the active compound in an aqueous medium in the presence of a non-toxic suspending agent such as sodium carboxymethyl-cellulose, and oily suspensions containing a compound of the present invention in a suitable vegetable oil, for example arachis oil.

The active compound may be formulated into granules with or without additional excipients. The granules may be ingested directly by the patient or they may be added to a suitable liquid carrier (for example water) before ingestion. The granules may contain disintegrants (for example a pharmaceutically acceptable effervescent couple formed from an acid and a carbonate or bicarbonate salt) to facilitate dispersion in the liquid medium.

Compositions of the invention suitable for rectal administration are the known pharmaceutical forms for such administration, for example, suppositories with cocoa butter or polyethylene glycol bases.

Compositions of the invention suitable for parenteral administration are the known pharmaceutical forms for such administration, for example sterile suspensions or sterile solutions in a suitable solvent.

Compositions for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as a mineral oil, petrolatum and/or a wax, for example paraffin wax or beeswax, together with a potential transdermal accelerant such as dimethyl sulphoxide or propylene glycol. Alternatively the active compounds may be dispersed in a pharmaceutically acceptable cream or ointment base. The amount of active compound contained in a topical formulation should be such that a therapeutically effective amount of the compound is delivered during the period of time for which the topical formulation is intended to be on the skin.

The compounds of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as a suspension or solution in a pharmaceutically acceptable oil of the compound to be infused for example in the form of a very sparingly water-soluble derivative such as a dodecanoate salt or ester or (b) solid in the form of an implanted support, for example of a synthetic resin or waxy material, for the compound to be infused. The support may be a single body containing all the compound or a series of several bodies each containing part of the compound to be delivered. The amount of active compound present in an internal source should be such that a therapeutically effective amount of the compound is delivered over a long period of time.

In some formulations it may be beneficial to use the compounds of the present invention in the form of particles of very small size, for example as obtained by fluid energy milling.

In the compositions of the present invention the active compound may, if desired, be associated with other compatible pharmacologically active ingredients.

The pharmaceutical compositions containing a therapeutically effective amount of a compound of formula I or a salt thereof may be used to treat depression, anxiety, psychoses (for example schizophrenia), tardive dyskinesia, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders, anorexia, cardiovascular and cerebrovascular disorders, migraine, non-insulin dependent diabetes mellitus, hyperglycaemia, constipation, arrhythmia, disorders of the neuroendocrine system, stress, prostatic hypertrophy, drug-induced extrapyramidal symptoms and spasticity in human beings. Whilst the precise amount of active compound administered in such treatment will depend on a number of factors, for example the age of the patient, the severity of the condition and the past medical history and always lies within the sound discretion of the administering physician, the amount of active compound administered per day is in the range 1 to 1000 mg preferably 5 to 500 mg given in single or divided doses at one or more times during the day.

The ability of compounds of formula I to interact with 5-hydroxytryptamine (5-HT) receptors has been demonstrated by the following test which determines the ability of the compounds to inhibit tritiated ligand binding to 5-HT receptors in vitro and in particular to 5-HT_{1A} receptors.

Hippocampal tissue from the brains of male Charles River CD rats weighing between 150-250 g were homogenised in ice-cold 50 mM Tris-HCl buffer (pH 7.7) when measured at 25° C., 1:40 w/v) and centrifuged at 30,000 g at 4° C. for 10 minutes. The pellet was rehomogenised in the same buffer, incubated at 37° C. for 10 minutes and centrifuged at 30,000 g at 4° C. for 10 minutes. The final pellet was resuspended in 50 mM Tris-HCl buffer (pH 7.7) containing 4 mM CaCl₂, 0.1% L-ascorbic acid and 10 μM pargyline hydrochloride (equivalent to 6.25 mg wet weight of tissue/ml) and used immediately in the binding assay. Aliquots (400 μl; equivalent to 2.5 mg wet weight of tissue/tube) of this suspension were added to tubes containing the ligand (50 μl; 2 nM) and distilled water (50 μl; total binding) or 5-HT (50 μl; 10 μM; non-specific binding) or test compound (50 μl; at a single concentration of 10⁻⁶ M or at 10 concentrations ranging from 10⁻¹¹-10⁻³ M). The ligand was [³H]8-hydroxy-2-(dipropylamino)tetralin ([³H]8-OH-DPAT) and the mixture was incubated at 25° C. for 30 minutes before the incubation was terminated by rapid filtration.

The filters were washed with ice-cold Tris-HCl buffer and dried. The filters were punched out into vials, scintillation fluid added and radioactivity determined by liquid scintillation counting. The percentage displacement of specific binding of the tritiated ligand was calculated for the single concentration (10⁻⁶ M) of test compound. Displacement curves were then produced for those compounds which displaced ≧50% of specific binding of the tritiated ligand at 10⁻⁶ M using a range of concentrations of the compound. The concentration which gave 50% inhibition of specific binding (IC₅₀) was obtained from the curve. The inhibition coefficient Ki was then calculated using the formula

$${K}_{i} = \frac{{IC}_{50}}{1 + ( [ ligand ] / {K}_{D} )}$$

in which [ligand] is the concentration of the tritiated ligand used and K_{D} is the equilibrium dissociation constant for the ligand.

The ability of compounds of formula I to interact with adrenoceptor binding sites has been demonstrated by the following test which determines the ability of the compounds to inhibit tritiated ligand binding toadrenoceptors in vitro and in particular α₁-adrenoceptors.

Whole cortical tissue from the brains of male Charles River CD rats weighing between 150-250 g were homogenised in ice-cold 50 mM Tris-HCl, pH 7.6 (at 25° C.; 1:40 w/v) and centrifuged at 1000 g at 4° C. for 10 minutes. The supernatant was centrifuged at 30,000 g at 4° C. for 10 minutes. The pellet was rehomogenised in 50 mM Tris-HCl, pH 7.6 (1:40 w/v) and centrifuged at 30,000 g at 4° C. for 10 minutes. The final pellet was resuspended in 50 mM Tris-HCl, pH 7.6 (equivalent to 12.5 mg wet weight of tissue/ml) and used immediately in the binding assay. Aliquots (400 μl; equivalent to 5 mg wet weight of tissue/tube) of this suspension were added to tubes containing the ligand (50 μl; 0.1 nM) and distilled water (50 μl; total binding) or phentolamine (50 μl; 5 μM; non-specific binding) or test compound (50 μl; at a single concentration of 10⁻⁶M or at 10 concentrations ranging from 10⁻¹¹-10⁻³M). The ligand was [7-methoxy-³H]prazosin and the mixture was incubated at 30° C. for 30 minutes before the incubation was terminated by rapid filtration.

The filters were washed with ice-cold Tris-HCl buffer and dried. The filters were punched out into vials, scintillation fluid added and radioactivity determined by liquid scintillation counting. The percentage displacement of specific binding of the tritiated ligand was calculated for the single concentration (10⁻⁶ M) of test compound. Displacement curves were then produced for those compounds which displaced ≧50% of specific binding of the tritiated ligand at 10⁻⁶ M using a range of concentrations of the compound. The concentration which gave 50% inhibition of specific binding (IC₅₀) was obtained from the curve. The inhibition coefficient Ki was then calculated using the formula

$${K}_{i} = \frac{{IC}_{50}}{1 + ( [ ligand ] / {K}_{D} )}$$

in which [ligand] is the concentration of the tritiated ligand used and K_{D} is the equilibrium dissociation constant for the ligand.

The ability of compounds of formula I to interact with dopamine receptors has been demonstrated by the following test which determines the ability of the compounds to inhibit tritiated ligand binding to dopamine receptors in vitro and in particular to the D₂ dopamine receptors.

Striatal tissue from the brains of male Charles River CD rats weighing between 140-250 g were homogenised in ice-cold 50 mM Tris-HCl buffer (pH 7.7 when measured at 25° C.) and centrifuged at 40,000 g for 10 minutes. The pellet was resuspended in Tris salts buffer (50 mM Tris-HCl buffer containing 120 mM NaCl, 5 mM KCl, 2 mM CaCl₂ and 1 mM MgCl₂ with the addition of 6 mM ascorbic acid; pH 7.7 when measured at 25° C.), and again centrifuged at 40,000 g for 10 minutes. The final pellet was stored at −80° C. Before each test the pellet was resuspended in Tris salts buffer (equivalent to 2 mg wet weight of tissue/ml). Aliquots (720 μl; equivalent to 1.44 mg wet weight of tissue/tube) of this suspension were then added to tubes containing the ligand (40 μl; 1 nM) and Tris salts buffer (40 μl; total binding) or spiroperidol (40 μl; 10 nM; non-specific binding) or test compound (40 μl; at a single concentration of 10⁻⁶M or at 6 concentrations ranging from 10⁻¹¹-10⁻⁴M). The ligand was tritiated (S)-sulpiride and the mixture was incubated at 4° C. for 40 minutes before the incubation was terminated by rapid filtration.

The filters were washed with ice-cold Tris-HCl buffer and dried. The filters were punched out in to vials, scintillation fluid added and were left for about 20 hours before being counted by scintillation spectrophotometry. The percentage displacement of specific binding of the tritiated ligand was calculated for the single concentration (10⁻⁶ M) of test compound. Displacement curves were then produced over a range of concentrations for those compounds which displaced ≧50% of specific binding of the tritiated ligand at 10⁻⁶M. The concentration which gave a 50% inhibition of specific binding (IC₅₀) was obtained from the curve. The inhibition coefficient Ki was then calculated using the formula

$${K}_{i} = \frac{{IC}_{50}}{1 + ( [ ligand ] / {K}_{D} )}$$

in which [ligand] is the concentration of the tritiated ligand used and K_{D} is the equilibrium dissociation constant for the ligand.

The Kᵢ values obtained in the above tests for 5-HT_{1A}, α₁ and D₂ binding for each of the final products of Examples 1, 2, and Comparative Example A are given in Table I below. It is clear from these data that compounds of the present invention have significantly less affinity for the α₁ adrenoceptor than compounds previously described. This is important as it is known in the art that α₁ receptor antagonism mediates serious side-effects such as hypotension, sedation and sexual dysfunction. Thus, compounds which interact preferentially with D₂ and 5-HT_{1A} receptors whilst having less interaction with α₁ receptors are advantaged.

TABLE 1

| Example | Ki (nM) value for | | |
|---|---|---|---|
| Number | 5-HT_{1A} | D₂ | α₁ |
| 1 | 12 | 11 | 101 |
| 2 | 37 | 25 | 573 |
| A | 22 | 44 | 53 |

Antagonism of Apomorphine-Induced Climbing in Mice

Groups of 10 male mice weighing 18-35 g (max. range 10 g) were treated with test compound or control vehicle by po administration. 30 minutes later, mice were injected subcutaneously with apomorphine (0.88 mg/kg). Immediately after the apomorphine injection the mice were placed in the test cages and the climbing behaviour of each mouse was assessed at 10 and 20 minutes on a simple 0-2 ranking scale.

The ED₅₀ values (dose causing 50% of the control score) for the test compounds and 95% confidence limits were calculated. ED₅₀ values are calculated as free base equivalents and are given in Table 2 alongside Comparative Example A. The compounds of the present invention are more potent orally than compounds previously described. More potent compounds are advantaged as they are less likely to induce systemic toxicological effects on organs which are not the therapeutic target.

TABLE 2

| Example | ED₅₀ (mg/kg) | [duration] |
|---|---|---|
| 1 | 3 | [>6 h] |
| 2 | 0.6 | [>4 h] |
| A | 5 | [>6 h] |

Processes for the preparation of compounds of formula I will now be described. These processes form a further aspect of the present invention. The processes are preferably carried out at atmospheric pressure. The substituents are as defined for formula I above unless otherwise stated.

Compounds of formula I in which R₂ represents H may be prepared by reacting a compound of formula III

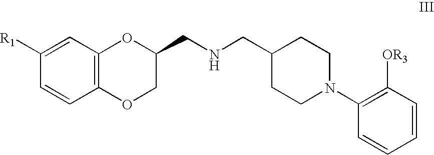

in which R₁ is as previously defined and R₃ is a C₁₋₆ alkyl group with a de-alkylating agent for example hydrobromic acid, pyridine hydrochloride or boron tribromide optionally in the presence of a suitable solvent or mixture of solvents, for example water or glacial acetic acid, or mixtures thereof, at a temperature in the range of 0-250° C.

Compounds of formula I in which R₂ represents an —O-acyl group may be prepared by acylating a compound of formula I in which R₂ represents H by methods known to those skilled in the art, for example by reaction with an anhydride or an acyl chloride, optionally in the presence of a solvent and optionally in the presence of a base. Preferably the methylamine nitrogen is protected before the acylation and then deprotected after the acylation by methods known to those skilled in the art.

The invention is illustrated by the following Examples which are given by way of example only. The final product of each of these Examples was characterised by one or more of the following procedures: gas-liquid chromatography; high performance liquid chromatography; elemental analysis, nuclear magnetic resonance spectroscopy and infrared spectroscopy.

### EXAMPLE 1

(S)-(−)-N-(7-Chloro-1,4-benzodioxan-2-ylmethyl)-1-(1-(2-methoxyphenyl)piperid-4-yl)-methylamine (4.54 g) (also known as (S)-(−)-N-(7-chloro-2,3-dihydro-1,4-benzodioxin-2-methyl)-1-(1-(2-methoxyphenyl)piperid-4-yl)methylamine) was prepared as described in WO 95/07274 and then dissolved in 48% hydrobromic acid (50 ml). The mixture was boiled under reflux for 18 hours. The mixture was cooled, poured into water (500 ml) and basified with concentrated aqueous ammonia solution. The resulting suspension was extracted with dichloromethane (3×200 ml). The combined organic extracts were washed with water (200 ml), dried over sodium sulphate and evaporated under reduced pressure to give (S)-(−)-2-{4-[N-(7-chloro-2,3-dihydro-1,4-benzodioxin-2-ylmethyl)aminomethyl]piperidino}phenol (or alternatively named as N-(7-chloro-2,3-dihydro-1,4-benzodioxin-2-ylmethyl)-(S)-(−)-1-[1-(2-hydroxyphenyl)-piperid-4-yl]methylamine) as a pale yellow oil which solidified on standing (m.p. 84-86° C.). The structure was confirmed by ¹Hnmr and infrared spectroscopy.

### EXAMPLE 2

(S)-(−)-1-[1-(2-Methoxyphenyl)piperid-4-yl]-N-(7-trifluoromethyl-2,3-dihydro-1,4-benzodioxin-2-ylmethyl)methylamine (0.45 g) prepared as described in WO99/62902, was heated with pyridine hydrochloride (10.0 g) at 180° C. for 6 hours. Further pyridine hydrochloride (5.0 g) was added and the mixture was heated for a further 6 hours at 180° C. and left to stand at ambient temperature for 72 hours. The resulting solid was dissolved in water (200 ml) and then basified to pH8 with concentrated aqueous ammonia solution. The mixture was extracted with ethyl acetate (2×100 ml). The combined organic extracts were dried, filtered and evaporated to give a brown oil which was purified by flash column chromatography to give (S)-(−)-2-{4-[N-(7-trifluoromethyl-2,3-dihydro-1,4-benzodioxin-2-ylmethyl)aminomethyl]piperidino}phenol {or alternatively named as (S)-(−)-1-[1-(2-hydroxyphenyl)piperid-4-yl]-N-(7-trifluoromethyl-2,3-dihydro-1,4-benzodioxin-2-ylmethyl)methylamine} as an oil.

### EXAMPLE 3

a) A solution of di-tert-butyl dicarbonate (0.14 g) in methanol (25 ml) was added dropwise to a stirred solution of (S)-(−)-N-(7-Chloro-1,4-benzodioxan-2-ylmethyl)-1-(1-(2-methoxyphenyl)piperid-4-yl)methylamine (0.5 g) in methanol (25 ml) at ambient temperature. The mixture was stirred at this temperature for 24 hours and then a further portion of di-tert-butyl dicarbonate (0.14 g) was added and the mixture was stirred at ambient temperature for 24 hours. The solvent was removed under reduced pressure to give a (S)-(−)-2-{4-[N-tert-butoxycarbonyl-N-(7-chloro-2,3-dihydro-1,4-benzodioxin-2-ylmethyl)-aminomethyl]piperidino}phenol as a yellow oil.

b) Sodium hydride (0.19 g of a 60% dispersion in mineral oil) was added to the solution of the product from a) (3.55 g) in tetrahydrofuran (100 ml) with stirring at ambient temperature under nitrogen. The mixture was stirred at ambient temperature for 5 minutes and then a solution of decanoyl chloride (1.38 g) was added dropwise. The mixture was stirred at ambient temperature for 16 hours and then poured into water (100 ml). The mixture was extracted with ethyl acetate to give an orange oil which was purified by flash column chromatography on silica using 15% ethyl acetate in petrol as the mobile phase. Appropriate fractions were combined and evaporated to give:(S)-(−)-2-{4-[N-tert-butoxycarbonyl-N-(7-chloro-2,3-dihydro-1,4-benzodioxin-2-ylmethyl)aminomethyl]piperidino}phenyl decanoate an oil which was identified by mass spectroscopy and ¹Hnmr.

c) Trifluoroacetic acid (7 ml) was added dropwise with care over a period of 5 minutes to an ice-cold stirred solution of the product from 3b) (3.0 g) in dichloromethane (100 ml). The mixture was allowed to warm to ambient temperature and stirred at this temperature for 24 hours. The mixture was poured into water (300 ml) and then basified with solid sodium bicarbonate. The organic layer was separated and the aqueous layer was extracted with dichloromethane. The combined organic layers were washed with water, dried over magnesium sulphate and evaporated to give (S)-(−)-2-{4-[N-(7-chloro-2,3-dihydro-1,4-benzodioxin-2-ylmethyl)aminomethyl]piperidino}-phenyl decanoate as an oil.

### COMPARATIVE EXAMPLE A

(S)-(−)-N-(7-Chloro-1,4-benzodioxan-2-ylmethyl)-1-(1-(2-methoxyphenyl)piperid-4-yl)methylamine (also known as (S)-(−)-N-(7-chloro-2,3-dihydro-1,4-benzodioxin-2-methyl)methylamine) was prepared as described in WO 95/07274.

### EXAMPLE A

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose are de-aggregated and blended. The mixture is filled into hard gelatin capsules, each capsule containing a unit dose or part of a unit dose of active compound.

b) Tablets

Tablets are prepared from the following ingredients.

| | Parts by weight |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch are de-aggregated, blended and the resulting mixture is granulated with a solution of the polyvinyl-pyrrolidone in ethanol. The dry granulate is blended with the magnesium stearate and the rest of the starch. The mixture is then compressed in atabletting machine to give tablets each containing a unit dose or a part of a unit dose of active compound.

c) Enteric Coated Tablets

Tablets are prepared by the method described in (b) above. The tablets are enteric coated in a conventional manner using a solution of 20% cellulose acetate phthalate and 3% diethyl phthalate in ethanol:dichloromethane (1:1).

d) Suppositories

In the preparation of suppositories, 100 parts by weight of active compound is incorporated in 1300 parts by weight of triglyceride suppository base and the mixture formed into suppositories each containing a therapeutically effective amount of active ingredient.

1. Compounds of formula I 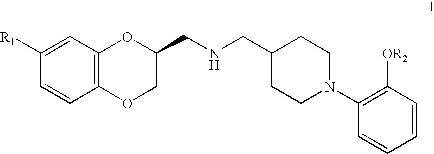
including pharmaceutically acceptable salts thereof in which R₁ represents halo or pseudohalo and R₂ represents H or an acyl group derived from a C₇-C₁₈ saturated aliphatic carboxylic acid; with the proviso that when R₁ is Cl or CF₃ and R₂ is H then these compounds are in isolated form.

2. A compound according to claim 1 which is (S)-(−)-2-{4-[N-(7-trifluoromethyl-2,3-dihydro-1,4-benzodioxin-2-ylmethyl)aminomethyl]piperidino}phenol and pharmaceutically acceptable salts thereof in isolated form.

3. A compound according to claim 1 which is (S)-(−)-2-{4-[N-(7-chloro-2,3-dihydro-1,4-benzodioxin-2-ylmethyl)aminomethyl]piperidino}phenol and pharmaceutically acceptable salts thereof in isolated form.

4. Pharmaceutical compositions comprising a therapeutically effective amount of a compound of formula I as claimed in any one of claims 1-3, together with a pharmaceutically acceptable diluent or carrier.

5. A method of treating depression, anxiety, psychoses, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders, anorexia, cardiovascular and cerebrovascular disorders, migraine, non-insulin dependent diabetes mellitus, hyperglycaemia, constipation,. arrhythmia, disorders of the neuroendocrine system, stress, prostatic hypertrophy, drug-induced extrapyramidal symptoms or spasticity which comprises the administration of a therapeutically effective amount of a compound of formula I as claimed in any one of claims 1-3 to a patient in need th reof.

6. A method as claimed in claim 5 for treating schizophrenia.

7. A method as claimed in claim 5 for treating anxiety.

8. A compound of formula I as claimed in any one of claims 1-3 for use as a medicament.

9. A compound of formula I as claimed in any one of claims 1-3 for use as a medicament for treating depression, anxiety, psychoses, tardive dyskinesia, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders, anorexia, cardiovascular and cerebrovascular disorders, migraine, non-insulin dependent diabetes mellitus, hyperglycaemia, constipation, arrhythmia, disorders of the neuroendocrine system, stress, prostatic hypertrophy, drug-induced extrapyramidal symptoms or spasticity.

10. The use of a compound of formula I as claimed in any one of claims 1-3 in the manufacture of a medicament for treating depression, anxiety, psychoses, tardive dyskinesia, Parkinson's disease, obesity, hypertension, Tourette's syndrome, sexual dysfunction, drug addiction, drug abuse, cognitive disorders, Alzheimer's disease, senile dementia, obsessive-compulsive behaviour, panic attacks, eating disorders, anorexia, cardiovascular and cerebrovascular disorders, migraine, non-insulin dependent diabetes mellitus, hyperglycaemia, constipation, arrhythmia, disorders of the neuroendocrine system, stress, prostatic hypertrophy, drug-induced extrapyramidal symptoms or spasticity.

11. Compounds according to claim 1 in which R₁ is Cl or CF₃ and R₁ is H.

12. A process for preparing compounds of formula I according to claim 1 comprising reacting a compound of formula II 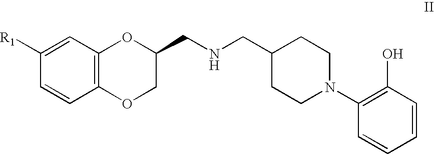
in which R₁ is as previously defined and R₂ is a C₁₋₆ alkyl group with a de-alkylating agent for example hydrobromic acid or pyridine hydrochloride.
